(19) United States
(12) Patent Application Publication
(10) Pub. No.: US 2007/0036731 A1
(43) Pub. Date: Feb. 15, 2007

(54) Topical Delivery with a Carrier Fluid

(75) Inventors: Jane C. Hirsh, Wellesley, MA (US); Ronald M. Gurge, Franklin, MA (US); Mark Hirsh, Wellesley, MA (US); Mark W. Trumbore, Westford, MA (US)

Correspondence Address: PATREA L. PABST;PABST PATENT GROUP LLP, 400 COLONY SQUARE, SUITE 1200, ATLANTA, GA, 30361, US

(73) Assignee: Collegium Pharmaceutical, Inc.

(21) Appl. No.: 11/464,100

(22) Filed: Aug. 11, 2006

Related U.S. Application Data
(60) Provisional application No. 60/708,286, filed on Aug. 13, 2005.
(60) Provisional application No. 60/797,186, filed on May 3, 2006.
(60) Provisional application No. 60/813,658, filed on Jun. 14, 2006.

(51) Int. Cl. A61K 31/7034; A61K 9/14; A61K 31/496; A61K 31/4178; A61K 31/4196

(52) U.S. Cl. 424/46; 514/35; 514/254.07; 514/171; 514/253.08; 514/383; 514/397

(57) ABSTRACT

Aerosol spray formulations capable of delivering high concentrations of active agent-containing materials and/or excipient are described herein. The formulation contains a carrier fluid, a propellant, and a therapeutic, prophylactic, consmeticeutical and/or inert solid suspended, dissolved, or dispersed in the formulation. The active ingredient may be any pharmaceutically active agent, but is preferably an antibiotic, an antihistamine, an anesthetic, an anti-inflammatory, and/or an astringent. In one embodiment, the active agent is an antifungal agent. In another embodiment, the active agent is a consmeticeutical. The active agent can optionally be dispersed on, or associated with, a carrier powder. The carrier fluid is a highly volatile silicone liquid, which evaporates in less than 10 minutes, preferably less than 5 minutes, after application of the formulation to the patient's skin. The formulation may also contain one or more pharmaceutically acceptable excipients such as antioxidants, stabilizers, perfumes, colorants, viscosifiers, emulsifiers, surfactants, and combinations thereof. The formulation can be packaged in a conventional aerosol spray can.

### CROSS-REFERENCE TO RELATED APPLICATIONS

Priority is claimed to U.S. provisional applications Ser. No. 60/708,286, filed Aug. 13, 2005, Ser. No. 60/797,186 filed May 3, 2006, and Ser. No. 60/813,658 filed in the U.S. Patent and Trademark Office on Jun. 14, 2006.

### BACKGROUND OF THE INVENTION

Topical dusting powders have been available for decades. These powders provide a simple means to dilute a potent active agent in a sufficient amount of diluent, for example talc, starch, zinc oxide or a combination of carriers, to provide a uniform dispersion of the therapeutic agent at the appropriate concentration. The selection of the active agent is dependent on the indication of use. The nature and concentration of the diluent are also important in providing therapeutic benefits by absorbing moisture or acting as a glidant. (Manual of Dermatologic Therapeutics, 6^{th} Ed., K. A. Arndt, K. E. Bowers; Lippincott Williams & Wilkins, Philadelphia, Pa. 19106; Ch. 14, pg 88-99.)

Dusting powders intended for a pharmaceutical use are typically packaged in a container with a shaker, from which the powder is sprinkled or dusted over the desired skin surface much like powdered sugar. Dusting powders often contain active agents, but these agents are typically only a small fraction of the weight of the preparation, for instance about 1% to 5%. In health-related uses, dusting powders are intended not only for the application of an active, but also to provide a glidant, such as talc, which typically comprises about 95% or so by weight of the preparation, and/or an absorbent, such as a non-soluble starch, to aid in the absorption of dampness and fluid secretions. This old product design is still used for many commercial pharmaceutical products, including, for example, Nystatin topical Powder USP 100,000 Units per gram, and Miconazole Nitrate 2% Topical Powder.

Dusting powder containers must be held upside down and shaken to dispense the powder. It can be difficult to control this method of dispensing, and to effectively target the intended site, especially in environments such as nursing homes where the patient may be immobile. Powders are often used to protect intertriginous areas, which include target sites such as folds or creases of skin, e.g. under breasts, in dewlaps, the buttocks, the groin and the peri-anal region, between the toes, and other areas. Powders can dry macerated skin and reduce friction by absorbing moisture. Hence, the quantity of powder available at the site is directly relevant to the ability of the preparation to absorb moisture.

For example, in present nursing home practice, the caretaker typically dispenses the powder into a mass in the target area and then spreads the powder with a gloved hand. This means that applications are not consistent, and sometimes excess powder needs to be removed from the patient, or from clothing or bed linen. The openings in the shaker can clog due to powder caking, atmospheric moisture, etc. This delivery system is unsuitable for moisture-sensitive pharmaceutical and medicinal ingredients, and is less than optimal for those ingredients that are sensitive to heat or light.

Dusting powders are a currently preferred method for topical delivery of antifungal agents. Topical infections involving fungi, whether based on semi-intact skin as in early forms of tinea, or in open skin situations such as wounds, can be treated topically, systemically, or both. Topical treatment is a first line of defense against mild, non-aggressive infection, and can be part of a combined topical/systemic strategy in open wounds. Generally, topical therapy will be preferred when effective, since many antifungal materials have undesirable systemic effects as equivalent dosages to topical applications. Moreover, topical administration is often preferred for completing the eradication of the causative organisms after initial control.

Treatment against fungi is usually a multi-day or multi-week course of administration. The medicinal preparation is usually applied to the site at least once a day, and often several times a day. However, as noted, available dosage formulations can be difficult to apply. Ointments and lotions require manual spreading, while dusting powders tend not to distribute evenly over the site, and do not adhere well. Many formulations require the use of gloves during application to prevent spread of the organisms. Nystatin is a well-known antibiotic with antifungal properties. A variety of brands of nystatin in formulations ready for topical application are available, but these are typically creams or ointments. One formulation for “dusting” applications, Nystop™, comprises nystatin adsorbed on talc, supplied in a squeeze bottle. “Zeasorb™” is a similar formulation. Squeezing the bottle sprays powder towards the skin. This is in principle a “hands-off” application, but as a practical matter, application is uneven, adherence to skin is poor, and rubbing to obtain an even and adherent coating may be required, which can be painful and has potential for contamination.

Aerosolized spray powders, also called dry spray powders, have been formulated and commercially available since the 1960's, and are well known in the art. For example, “Aerosols: Science and Technology”, (H. R. Shepard (Ed.), 1961, Interscience Publishers/J Wiley; Chapter 10) describes several procedures for formulating aerosol powders. Concentrations of solid ingredients are less than 10%, typically less than about 5%, most often in the range of 1-2% or less.

Aerosolized spray powder technology has not changed greatly in the last several decades. In a sampling of contemporary commercially available aerosolized spray powders, it was found that the solid ingredients, usually containing between 1 and 2% active ingredients plus other materials, typically are less than about 10% of the fill weight, while propellants generally make up 90 to 95% of the product weight. Generally these spray powder aerosols contain some combination of SD alcohol 40 (high grade purified ethanol) and isobutane to increase pressure along with an appropriate valve and stem apparatus to allow the solids to be expelled without clogging. Unfortunately, this frequently results in a cloud of airborne dust, also known as “bounce off”. The properties of several commercially available aerosol spray powders are described in Table 1. Details on testing are described in Example 2, below.

TABLE 1
Current Commercial Spray Preparations

| | Brand: | | |
|---|---|---|---|
| | Lotrimin ™ | Tinactin ™ | Neosporin ™ |
| Active ingredient | 2% miconazole | 1% tolnaftate | 2% miconazole |
| | nitrate | | nitrate |
| Propellant: | isobutane | isobutane | isobutane/propane |
| Dispensed weight: | 1.9918 g | 2.0505 g | 2.0898 g |
| Dry (powder) wt | 0.1395 | 0.1019 | 0.2216 |
| (after 10 minutes) | | | |
| % solids | 7.0% | 5.0% | 10.6% |
| % volatiles | 93% | 95% | 89.4% |

Experiments were conducted based on the above formulation utilizing the same propellant and valve and stem assembly, except that the dry (powder) weight was increased to 35%. Even with vigorous shaking, the material rapidly clogged and would not deliver repeated sprays.

One important aspect of a spray delivery system is the composition of the propellant and any liquid materials in the composition. Spray vehicles typically include silicones, such as dimethicone, simethicone, or cyclomethicone. These silicones and other low MW polysiloxanes, including oligomeric cyclosiloxanes, may be obtained by fractional distillation or standard silicones, or prepared synthetically. While these materials can be liquid, they are not typically very volatile, and tend to be perceived as “wet” when used as a spray. Such materials have been used for extended-wear cosmetics (e.g. U.S. Pat. No. 6,887,859) and as a low volatility carrier of antifungals (U.S. Pat. No. 5,262,150), but their poor volatility prevents them from being used to deliver high solids formulations.

Another important aspect of a spray delivery system is the concentration of the propellant. As noted above, most sprays contain 90% or more by weight propellant. Delivery of a higher concentration of solid materials (i.e., lower concentration of propellant) would make such systems more efficient. Propellant concentration has become a more significant issue since the banning of chlorofluorocarbon (CFC) propellants.

Current aerosol spray powders are not designed to dispense more than a few percent by weight of deliverable ingredients, which may reflect an inherent limitation of the formulation. This makes the delivery of significant amounts of talc or other carrier or glidant quite difficult, and may explain the persistence in the market of the simple dusting powders despite their difficulties in accurate dosing, and general messiness. There exists a need for an aerosol spray powder formulation containing a high percentage of solids.

It is therefore an object of the present invention to provide spray formulations containing high percentages of solids.

It is another object of the present invention to provide spray formulations that are efficient and do not utilize CFC propellants.

It is a further object of the present invention to provide spray formulation with excellent dispersion and uniformity of application.

### SUMMARY OF THE INVENTION

Aerosol spray formulations capable of delivering high concentrations of active agent-containing materials and/or excipient are described herein. The formulation contains a carrier fluid, a propellant, and a therapeutic, prophylactic, cosmeticeutical (referred to herein as “active”) and/or inert solid (referred to herein as “inert”) suspended, dissolved, or dispersed in the formulation. The active ingredient may be any pharmaceutically active agent, but is preferably an antibiotic, an antihistamine, an anesthetic, and anti-inflammatory, and/or an astringent. In one embodiment, the active agent is an antifungal agent. In another embodiment, the active agent is a cosmeticeutical. The active agent can optionally be dispersed on, or associated with, a carrier powder. The carrier fluid is a highly volatile silicone liquid, which is somewhat less volatile than the propellant, which evaporates in less than 10 minutes, preferably less than 5 minutes, after application of the formulation to the patient's skin. The formulation may also contain one or more pharmaceutically acceptable excipients such as antioxidants, stabilizers, perfumes, colorants, viscosifiers, emulsifiers, surfactants, and combinations thereof. The formulation can be packaged in conventional aerosol spray can.

Any USP grade of the active or inert, the carrier, or the propellant is potentially acceptable. Finer powder grades of the “dry” ingredients, or of their complexes, are preferred. The preferred particle size range is 0.01-2000 microns. To minimize plugging of the nozzle of the aerosol container, uniform grades of the “dry” ingredients are preferred. The preferred size grade will depend on the valve and stem orifice diameter selected.

The capacity of an aerosol spray for delivering high concentrations of drug-containing materials or of excipients can be greatly improved by inclusion of the “carrier fluid” in the formulation, along with the aerosol propellant. Use of the carrier fluid solves a significant formulation problem in aerosolizing true dusting powders and other particulates and can increase the deliverable solids loading of the formulation from a few percent to tens of percent. Moreover, the carrier fluid appears to improve control of the dispersion of the product during spraying, confining the product to a narrow spray cone with reasonably uniform distribution. The carrier fluid can eliminate the necessity of utilizing SD alcohol (ethanol) in combination with isobutane to increase vapor pressure. Additionally the carrier fluid imparts a greater cooling and refreshing effect due to its sensory aspects on the skin. Preferred carrier fluids are highly volatile silicone liquids, somewhat less volatile than the propellant, that evaporate in less than 10 minutes, preferably less than 5 minutes, on a patient's skin. These aerosol spray delivery systems are especially useful for topical delivery of a highly-active drug dispersed on a high-surface area carrier.

### DETAILED DESCRIPTION OF THE INVENTION

### Definitions

“Topical”, as used herein, refers to the application to any surface accessible to a reagent applied as an aerosol.

“Volatile”, as used herein, refers to a material having a boiling point in dry air at atmospheric pressure below about 250° C., preferably below 225° C., more preferably below 200° C., and most preferably below 180° C.

“Antifungal”, as used herein, refers to a pharmaceutically active ingredient having antibiotic activity against fungi (including yeasts).

A “carrier”, as used herein, refers to a semi-solid or solid material that can be used to dispense an antifungal or other pharmaceutical agent. Lotions, creams, ointments and gels are examples of semi-solid carriers. If the “carrier” is a solid material, it is referred to as a “carrier powder”.

A “carrier fluid”, as used herein, refers to a liquid, compatible with the propellant and with the active agent, which remains liquid while the propellant evaporates. The carrier fluid itself then evaporates, sufficiently to immobilize a powder or a drug, over a period of no more than about ten minutes, preferably no more than about five minutes, more preferably no more than about three minutes after application to the skin of a patient.

A “high solid content” and “high percentage of solids”, as used herein, refer to a level of solid material in the formulation that is above 10% and preferably is in the range from about 15% to about 75% (w/w).

I. Formulations

A. Carrier Fluids

The carrier fluid contains one or more highly volatile silicones (“HV silicone” or “silicone oil”) in a concentration from about 50% to about 100% by weight of the carrier fluid. As used herein, the term “highly volatile silicone” includes, but is not limited to, commercial grades of hexamethyldisiloxane, octamethyltrisiloxane, and mixtures thereof. In one embodiment, the HV silicone is Dow Corning Q7-9180 Silicone Fluid, having a viscosity of about 1 centiStoke and a boiling point of about 153° C.), Dow Corning Q7-9180 Silicone Fluid having a viscosity of about 0.65 cSt and a boiling point of about 100° C., or combinations thereof. The key features of this HV silicone material as a carrier fluid are believed to be its fast evaporation rate (faster than ethanol) combined with its low level of imitation of skin. Its intermediate polarity, between the polarities of the powder and the propellant, may also be important. Note that this material is different from conventional liquid silicones, such as dimethicone, simethicone, or cyclomethicone (which includes oligomeric cyclosiloxanes).

Other highly volatile liquids may be used in combination with HV silicone to form a carrier fluid. Liquids acceptable as excipients, which can come in contact with skin, and have a boiling point in the approximate range of 10° C. to 200° C., more preferably a range of about 30-160° C., may be used. The liquid(s) should be selected so that they substantially evaporate, at least sufficiently to render the powder and any carrier immobile on the skin, in less than about 10 minutes at skin temperature, for example 25-30° C. Shorter drying times are preferred, for example 5 minutes, more preferably 3 minutes, most preferably 1 minute or less. Examples include, but are not limited to, lower alcohols, such as methanol, ethanol, and propanols; glycols, such as ethylene glycol and propylene glycol; lower ketones, such as acetone, MEK, and cyclohexanone; lower alkyl esters, such as methyl formate and ethyl acetate; ethers, such as diethyl ether, and mixtures thereof. Medium alkanes, such as linear, branched, and cyclic C₅-C₁₂ alkanes and small, inert volatile compounds (e.g., dioxane, N-methyl pyrrolidone, dimethylformamide, dimethylsulfoxide, and similar molecular weight compounds) may also be used, alone or mixed with more volatile compounds. Halogenated volatile compounds having low flammability and no toxicity are preferred.

Potentially useful silicones for inclusion in a carrier fluid containing 50% or more of the HV silicones include decamethyltetrasiloxane, dodecylmethylpentasiloxane, tetradecamethylhexasiloxane, hexadecamethylheptasiloxane, octamethylcyclotetrasiloxane, decamethylcyclopentasiloxane, dodecamethylhexasiloxane, and heptamethyl-3-(trimethylsilyloxy)trisiloxane. Mixtures of volatile liquids may also be used as components of carrier fluids.

The concentration of the carrier fluid is from about 10% to about 45% by weight of the composition, preferably from about 10% to about 25% by weight of the composition. The carrier fluid is selected to be a lqiuid at room temperature (approximately 25° C.), to be volatile, and to evarporate rapidly at body temperature (approximately 37° C.). In addition, the carrier fluid is preferably non-irritating to tissue, including macerated tissue. It is believed that an effective carrier fluid, such as HV silicone, is effective in producing an evenly dispersed powder coating on the skin because it evaporates significantly more slowly than the propellant, and so effectively carries the powder to the skin surface (reducing bounce off) and temporarily adheres the powder to the skin. Without being bound by any one particular theory, it is believed that the carrier fluid adheres the powder to the skin by surface tension, possibly forming a transient liquid-like layer. However, the carrier fluid must evaporate rapidly so that the preparation dries rapidly to an immobilized powder, i.e., is not “runny”. The carrier fluid also may assist in the dispersion of solid ingredients, for example by shaking, that is typically required before beginning a spray treatment.

B. Propellants.

A pharmaceutically-acceptable propellant comprises from about 5% to about 60% of the formulation. Suitable propellants include, but are not limited to, conventional aerosol propellants used with pharmaceutical formulations. Exemplary propellants include, but are not limited to, alkane and alkylene gases, such as pentane, butene, butane, isobutane, and mixtures thereof; and hydrofluoroalkanes (“HFA”), also known as hydrofluorocarbons (“HFC”). In one embodiment, at least part of the propellant is a hydrofluoroalkane. HFA propellants are desirable because of their high volatility and low flammability, combined with their relatively low ozone-destruction potential. The HFAs typically have significantly greater propelling power per unit volume compared to the alkanes, and thus adequate propulsion can be obtained at lower fractional percentages of HFA propellants in the formulation. Any HFA approved for medicinal use, presently or in the future, can be used. In one embodiment, HFA 134a (tetrafluoroethane) is the propellant. In another embodiment, HFA 227 (heptafluoropentane) is the propellant. A conventional standard aerosol propellant, A46, containing alkanes (butane, propane and isobutane), has also been tested, and works under appropriate conditions as described below. It is expected that other conventional alkane propellants will be suitable, in many cases as higher percentages of the total charge, to give adequate pressure.

The propellant can also contain, in part, compressed gasses such as nitrogen, carbon dioxide, argon or air. Mixtures of any of these can be used. Co-solvents, including but not limited to, alcohols, especially glycols such as diethylene glycol, dipropylene glycol, and other non-stinging alcohols, can be used to regulate the pressure in the container. It is preferred to have pressures in the container comparable to those found when using HFAs as propellants, although this may require higher pressures of alkanes at filling. Experimentation establishes that SD alcohol (ethanol) can comprise a large percentage of the carrier fluid, and delivers consistently high dry powder weights. Substitution of alcohol into the formulation is dependent on the particular active drug and its intended therapeutic use.

C. Therapeutics, Prophylactics and Cosmeticeuticals

The formulation can be used for delivery of one or more therapeutic, prophylactic, cosmeticeutic, or inert agents.

Examples of therapeutics include, but are not limited to, antibiotics and antifungals. Antibiotics are generally used to treat or prevent infectious diseases caused by bacteria while antifungal agents are generally used to treat infections caused by fungi.

Antifungals include, without limitation, amphotericin, amorolfine, bacitracin, bifonazole, bromochlorosalicyanilide, buclosamide, butenafine, butoconazole, candicidin, chlordantoin, chlormidazole, chlorphenesin, chlorxylenol, ciclopirox olamine, cilofungin, clotrimazole, croconazole, eberconazole, econazole, enilconazole, fenticlor, fenticonazole, fluconazole, flucytosine, griseofulvin, hachimycin, haloprogin, hydorxystilbamine isethionate, iodochlorohydroxyquinone, isoconazole, itraconazole, ketoconazole, lanoconazole, luflucarban, mepartricin, metroconazole, metronidazole, miconazole, naftifine, natamycin, neomycin, neticonazole, nifuroxime, nystatin, omoconazole, oxiconazole, pentamycin, posaconazole, propionic acid, protiofate, pyrrolnitrin, ravuconazole, saperconazole, selenium sulfide, sertaconazole, sulbentine, sulconazole, terbinafine, terconazole, tioconazole, tolciclate, tolnaftate, triacetin, undecenoic acid, voriconazole, and their pharmaceutically acceptable salts and esters. A presently-preferred antifungal is nystatin. Other preferred antifungals include miconazole, clotrimazole, terbinafine, tolnaftate and butenafine. Antiseptic materials having antifungal activity, such as zinc undecylate, may be used. In one embodiment, the antifungal agent is nystatin.

Other topically effective medications useful in the present invention include iodine, cadexomer, cadexomer iodine, silver, and various silver salts.

Any topically effective medication can be delivered, including, but not limited to, antihistamines, local anesthetics, and anti-inflammatory medications.

Representative cosmeticeuticals include, but are not limited to, hydrating agents, exfoliants, colorizers, fragrances, lubricants and alpha/beta hydroxyl-acids.

Representative inert materials include, but are not limited to, materials such as talc and other glidants. These are also described in more detail below as powders and powder carriers.

D. Carriers

A carrier is optional. The use of carriers can be helpful in obtaining even dispersion of the drug, and in visulaizing the spray as it is being applied to the patient. Carriers are also useful for highly active drugs to prevent localized overdosing. Carrier materials that are sufficiently finely divided to pass through the nozzle of the aerosol can be used. For example, powders can be selected in grades having maximal diameters below 50 microns, preferably below 10 microns, more preferably below 1 micron. Carriers are selected to be USP grade (or equivalent in other jurisdictions). Carriers may be sieved to eliminate oversize particles if necessary.

Examples of semi-solid carriers include, but are not limited to lotions, creams, ointments, and gels. Semi-solid carriers can be prepared as described in “Remington: The Science and Practice of Pharmacy” (20^{th} Edition, Lippincott Williams & Wilkins).

Examples of solid carriers include, but are not limited to, one or more of a crystalline or amorphous particulate non-organic compound, an inorganic salt, an inorganic/organic salt, an insoluble natural, synthetic, or semi-synthetic polymer, a charcoal, an organic resin, and mixtures thereof. Crystalline or amorphous particulate non-organic compounds include, without limitation, silicas, aluminas, aluminosilicates, borosilicates, titanias, and similar compounds. Inorganic salts include, but are not limited to, salts of silicates, aluminosilicates, borosilicates, carbonates, sulfates, aluminates, titanates, phosphates and combinations thereof, and particularly divalent or trivalent cationic salts of such anions, including calcium, magnesium, and zinc salts, inorganic/organic salts, such as calcium succinate, hydrates of any of these, including, for example, talc; bentonite; and calamine and other oxides of zinc, iron, and other transition metals.

Organic particulates include, without limitation, non-soluble celluloses, such as microcrystalline cellulose, ethyl cellulose, and methyl cellulose; insoluble starches; insoluble organic gums; other insoluble polysaccharides and derivatives thereof, such as chitin; insoluble synthetic and semisynthetic organic polymers; and other insoluble particulate materials, including powdered charcoals and organic resin particles; and mixtures of such materials. Examples of such materials include aluminum starch octenylsuccinate (Dri Flo Pure 28-1850; National Starch) (see Examples below), and materials such as modified corn starch, tapioca starch, polyacrylates and polyaccrylamides (e.g. Dermactyl 79, National starch), and similar materials. Carriers preferably have no significant effect on intact skin, and more preferably have no significant effect on broken skin, and are preferably USP grade or equivalent.

E. Other Ingredients

The formulation may optionally include onr ore pharmaceutically acceptable excipients found in tropical formulations, including without limitation, antioxidants, colorants, perfumes, vicostifying agents cofactors for drugs, penetration enhancers, surfactants, emulsifiers and cosolvents. The concentration of the one or more pharmaceutically acceptable excipients is generally less than about 10% of the composition, but could be higher, for example up to about 20% depending on the desired formulation. Higher levels of excipients may tend to reduce the improvement in levels of carrier and drug that are obtained by use of the carrier fluid.

II. Method of Manufacture

The compositions described herein are typically prepared by mixing the ingredients in an explosion proof kettle which is modified to control the loss of the volatile materials. The resultant slurry is filled into aerosol cans. Valves are placed on the cans, crimped and the propellant is charged. Alternatively, a pharmaceutically active agent is places into the aerosol container, a semi-solid or solid carrier is optionally added, followed by the addition of a carrier fluid. Valves are placed on the cans crimped and the propellant is charged.

III. Method of Administration

In one embodiment, the formulation is shaken until all particulate material is suspended in the carrier fluid and propellant, then a valve is opened so that the formulation will spray out through the opening to the site of intended application. The container is typically is typically moved to insure a uniform dispersion at the site of application.

The present invention will be further understood by reference to the following non-limiting examples.

### EXAMPLES

### Example 1

Nystatin-Talc Formulations.

The mystatin-talc complex is well known for use in treatment of topical fungal infections. However, the uniformity of dispersion of the basic mystatin-talc is poor, and is variable depending on the relative humidity at the time of application. The ratio of the talc to the nystatin is variable, but a ratio of about 100,000 USP units of nystatin per gram of talc is preferred, because there is extensive clinical experience available with this formulation ratio. The nystatin used in this example had an activity of about 5600 units/mg, and was present in the range of about 20 mg/gm talc, or about 2% by weight, giving the standard 100,000 units/gram talc. The nystatin is physically mixed with the talc to obtain the diluted powder mixture, but no additional procedure is used.

The three examples described in Table 2 show the feasibility of the formulation, and illustrate some of the variables encountered in its optimization. Percentages are by weight. The silicones are HV silicones.

TABLE 2
Formulation Examples A, B, and C

| INGREDIENT | A (%) | B (%) | C (%) |
|---|---|---|---|
| Talc, USP | 35.7 | 23.8 | 35.7 |
| Nystatin USP | 0.65 | 0.44 | 0.65 |
| 5574 U/mg | | | |
| Silicone fluid | 36.4 | 48.5 | 36.4 |
| (viscosity) | (0.65 cSt) | (0.65 cSt) | (1.0 cSt) |
| HFC-134a propellant | 27.3 | 27.3 | 27.3 |
| Total: | 100 | 100 | 100 |
| Spray properties: | Even, quick dry | Even, slow | Even, slow |
| | | dry | dry |

By diminishing the content of nystatin/talc and increasing the concentration of HV silicone at constant propellant concentration, a range of sprayable formulations has been found that have variable drying times. Formulation A had the fastest drying time. Formulation C with a higher viscosity HV silicone (octamethyl trisiloxane, 1.0 sCt) has a drying rate similar to formulation B, albeit slower than formulation A. The drying times of formulations B and C were within acceptable ranges.

### Example 2

Comparison of Formulations of Example 1 with Commercially Available Products.

Formulation A was compared with the commercially-available dusting powder nystatin/talc formulation. Application of the commercial product to the skin, by puffing the powder out of a bottle onto a volunteer's arm, resulted in an application of powder that was clumped and did not adhere well to the skin. Trickling of the powder down the arm was observed and there was some “cloudiness” in the air. Formulation A, described In Example 1, was sprayed onto the skin of the other arm. The HFA and silicone evaporated, largely within about 15 seconds or less, depositing an even layer of powder distributed in the region of spraying. The increased evenness and degree of control were evident. The powder layer was more resistant to disturbance. No “flyaway” or cloud, or extended pattern of distribution, was seen during application of the preparation.

In another experiment, the three commercial products described in Table 1 were qualitatively compared in spray pattern with formulation A described in Example 1. The materials were sprayed on a dark surface from a fixed distance (6 inches) and the dried spots were photographed. The apparent diameter of the spot left by each of the commercial preparations was about 5 inches, and after selection of an appropriate nozzle, the pattern achieved with a preparation identical to the preparation of formulation A was also about 5 inches. However, the spot of formulation A had a sharp perimeter, while there was a significant “halo” around the spots from the commercial preparations. Thus, formulation A appears to be superior in both material loading and in precision and evenness of delivery.

The time required for the powder layer to dry was approximated by observing the rate of weight loss after applying a sample of the formulation to a film on a balance. Approximately 25% of the carrier, presumable mostly silicone, had evaporated after 30 seconds at about 30° C. Equilibrium weight was reached after about 2.5 to 3 minutes. (Because of its evaporation rate, it is believed that little if any of the HFC 134a was present at 30 seconds.)

### Example 3

An Alternative Nystatin-Talc Formulation.

An alternative Nystatin Talc Aerosol formulation was prepared (see Table 3). Nystain was mixed with talc and then with silicon fluid. The resultant slurry was placed into the aerosol container and charged with propellant.

TABLE 3
Alternative Nystatin-Talc Formulations

| Ingredient | Supplier | Lot # | w/w % |
|---|---|---|---|
| Talc, USP | Spectrum | QE0177 | 35.49 |
| Nystatin, USP (5574 U/mg) | Spectrum | TN0176 | 0.87 |
| Q7-9180 Silicone Fluid | Dow Corning | 002187684 | 36.36 |
| (0.65 cSt) | | | |
| HFA-134A | Dupont | 0504FF0034 | 27.28 |
| Total | | | 100.00 |

### Example 4

Nystatin/talc Experiments—Non-HFC Propellants

The use of non-HCF propellants was tested, and the effects of the omission of the carrier fluid were examined.

Procedure:

10 g of Talc/Nystatin powder (130,000 U/g) was blended with 6.5 g of Dow Corning Q7-9180 Silicone Fluid (1.0 CST) using a mortar and pestle. The slurry was pumped into an aerosol can using a polypropylene syringe. Next, 6.5 g of Dow Corning Q7-9180 Silicone Fluid (0.65 CST) was added to the can. A continuous ferrule valve was crimped onto the can and propellant was added according to table 4 below.

TABLE 4
Nystatin-Talc Formulations Containing Non-HFC Propellants

| | Formulation | | | |
|---|---|---|---|---|
| | A | B | C | D |
| Nystatin/Talc (130,000 U/g)(g) | 10 | 10 | 10 | 10 |
| Q7-9180 Silicone Fluid, | 6.5 | 6.5 | 6.5 | |
| 1.0 CST (g) | | | | |
| Q7-9180 Silicone Fluid, | 6.5 | 6.5 | 6.5 | |
| 0.65 CST (g) | | | | |
| 134A Propellant (g) | 7.5 | | | 7.5 |
| A-46 Propellant (g) | | 7.5 | 11.3 | |
| Total (g) | 30.5 | 30.5 | 34.3 | 17.5 |
| Nystatin/Talc (130,000 U/g)(%) | 32.79 | 32.79 | 29.15 | 57.14 |
| Q7-9180 Silicone Fluid, | 21.31 | 21.31 | 18.95 | |
| 1.0 CST (%) | | | | |
| Q7-9180 Silicone Fluid, | 21.31 | 21.31 | 18.95 | |
| 0.65 CST (%) | | | | |
| 134A Propellant (%) | 24.59 | | | 42.86 |
| A-46 Propellant (%) | | 24.59 | 32.94 | |
| Total (%) | 100 | 100 | 100 | 100 |

Formulation A, utilizing HFC 134A propellant, was easily expelled from the can in the form of a powder spray with a consistent spray pattern. Formulation B, utilizing A-46 propane/isobutane propellant (equivalent to % weight of 134A in formulation A), did expel from the can. However, after a few actuations, formulation B exhibited clogging and an erratic spray pattern. Formulation B clogged completely after about 4 actuations. Formulation C, utilizing A-46 propellant equivalent to 1.5 times the amount of 134A in formulation A, was easily expelled from the can in the form of a powder spray with a consistent spray pattern. The entire contents of the can containing Formulation C were dispensed with no clogging. Formulation D was prepared with HFC 134a propellant, but without the use of silicone carrier fluids. Upon actuation, propellant expelled from the can, however, no powder was dispensed. Further actuations demonstrated an intermittent powder dispensing pattern which ultimately led to complete clogging. This demonstrated the necessity of the silicone fluids in the formulation.

These results demonstrate that the formulation requires an optimal pressure to deliver an effective amount of product throughout the lifetime of the can (without failure). HFC 134A propellant is the most effective; however, the use of A-46 propellant at higher concentrations can also deliver the aerosol powder. Furthermore, other hydrocarbon propellant blends (i.e. A-70, approximately 51% propane and 38% isobutane) may also be effective propellants relative to the pressure they would exert (A-70=70 psi@70° F., 134A=71.1 psi@70° F.).

### Example 5

Use of Non-Talc Carriers.

A formulation was prepared containing 2% by weight miconazole nitrate (an antifungal); 35% aluminum starch octenylsuccinate (DryFlo AF Pure (28-1855); 36% HV silicone (0.65 cSt); and 27% HCF 134a propellant. The valve was a 0.041 inch NM13 and stem was 2×0.020 inch.

When the formulation was shaken and sprayed, it did not clog, but exhibited some bounce-off. When the silicon fluid was changed to a ratio of 50:50 0.65 cSt to 1.0 cSt, the bounce-off decreased, and the spray pattern was initially slightly “wetter”, but very even. This shows that non-talc carriers are effective, and that the detailed pattern of laying-down of the aerosolized ingredient on the skin can be controlled in part by adjustment of volatile HV silicone viscosity.

### Example 6

High Solids-Aerosolized Ointment

A high-solids-containing aerosolized ointment formulation was prepared from the ingredients in Table 5.

TABLE 5
Composition of a High Solids-Aerosolized Ointment

| Ingredient | w/w % |
|---|---|
| Zinc Oxide | 16 |
| Talc | 8 |
| Petrolatum | 16 |
| Q7-9180 Silicone Fluid, 0.65 cSt | 30 |
| HFA-134A | 30 |
| Total | 100 |

Petrolatum was melted at 70° C. and added to a mixture of Zinc Oxide and Talc. The formulation was mixed until uniform and allowed to cool to room temperature with mixing. 40 g of the ointment was charged into an epoxy-lined aluminum aerosol can. 30 grams of HV Silicone (0.65 cSt) was added to the can, then an aerosol valve was added, and the can was crimped and charged with 30 g of HFA-134A. The aerosolized ointment formulation was then shaken and sprayed to give an even and substantive application of the high solids ointment. The formulation sprayed without evidence of clogging.

### Example 7

High Solids-Aerosolized Gel

A high solids containing aerosolized gel formulation was prepared from the ingredients in Table 6.

TABLE 6
Composition of a High Solids-Aerosolized Gel

| Ingredient | w/w % |
|---|---|
| Part A | |
| Mineral Oil&ethylene/propylene/styrene copolymer& | |
| butylenes/ethylene/styrene copolymer (Versagel M750) | 29.84 |
| Isopropyl Isostearate | 5.25 |
| Propylene glycol isoceteth-3 acetate | 4.00 |
| Part B | |
| Isopropylisostearate | 0.80 |
| Propylparaben | 0.04 |
| Part C | |
| Fragrance | 0.07 |
| Zinc Oxide | 20.00 |
| Part D | |
| HV Silicone 0.65 cSt | 20.00 |
| HFA-134A Propellant | 20.00 |
| Total | 100 |

In the procedure, part A was mixed at 70° C. until uniform and part B was heated to 60° C. until clear. Part A was added to part B at 70° C. and mixed until uniform. The formulation was then cooled at 45° C. and part D was added and mixed until uniform.

60 g of the ointment was charged into an epoxy-lined aluminum aerosol can. 20 grams of HV Silicone (0.65 cSt) was added to the can and an aerosol valve was added. The aerosol can was crimped and charged with 20 g of HFA-134A propellant. The aerosolized gel formulation was then shaken and sprayed to give an even and substantive application of the high solids gel. The formulation sprayed without evidence of clogging and demonstrated an initial foam followed by a quick-breaking action resulting in even application to the surface.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim:

1. A formulation for topical administration, the formulation comprising:
a particulate material,
a volatile carrier fluid comprising one or more high volatility silicones, and
an aerosol propellant.

2. The formulation of claim 1, comprising a pharmaceutically acceptable carrier.

3. The formulation of claim 2 wherein the carrier is a particulate material selected from the group consisting of crystalline or amorphous particulate non-ionic non-organic compounds, inorganic salts, organic salts, insoluble natural, synthetic, or semi-synthetic polymers, charcoal, organic resins, and mixtures thereof.

4. The formulation of claim 3 wherein the carrier is an inorganic or organic salt of an anion selected from the group consisting of silicates, borosilicates, carbonates, sulfates, aluminates, aluminosilicates, titanates, phosphates, and combinations thereof.

5. The formulation of claim 1 wherein the carrier fluid contains one or more highly volatile silicones in a concentration from about 50% to about 100% by weight of the carrier fluid.

6. The formulation of claim 1 wherein the carrier fluid comprises at least one volatile compound which, when administered to the skin, evaporates sufficiently in less than about 10 minutes at body surface temperature to render the particulate material immobile on the skin.

7. The formulation of claim 6 wherein the particulate material is evenly dispersed in the carrier fluid.

8. The formulation of claim 1 wherein the carrier fluid has a boiling point in the range of about 10° C. to about 160° C.

9. The formulation of claim 6 wherein the carrier fluid is selected from volatile organic silicones, lower alcohols, glycols, lower ketones, lower alkyl esters and ethers, medium alkanes and unsaturated alkanes, dioxane, N-methyl pyrrolidone, dimethylformamide, dimethylsulfoxide, halogenated derivatives thereof, and mixtures thereof.

10. The formulation of claim 1 wherein the carrier fluid is a volatile organic silicone selected from the group consisting of hexamethyldisiloxane, octamethyltrisiloxane, and combinations thereof.

11. The formulation of claim 1 wherein the aerosol propellant is selected from the group consisting of alkane gases, alkylene gases, volatile hydrofluoroalkanes, and combinations thereof.

12. The formulation of claim 11 wherein the aerosol propellant further comprises one or more of compressed gases and co-solvents.

13. The formulation of claim comprising an active agent.

14. The formulation of claim 1 wherein the particulate material comprises an active agent selected from the group consisting of antibiotics, antihistamines, antifungals, local anesthetics anti-inflammatory medications, and combinations thereof.

15. The formulation of claim 14 wherein the particulate material comprises an antibiotic.

16. The formulation of claim 15 wherein the antibiotic is an antifungal and comprises one or more of amphotericin, amorolfine, bacitracin, bifonazole, bromochlorosalicyanilide, buclosamide, butenafine, butoconazole, candicidin, chlordantoin, chlormidazole, chlorphensin, chlorxylenol, ciclopirox olamine, cilofungin, clotrimazole, croconazole, eberconazole, econazole, enilconazole, fenticlor, fenticonazole, fluconazole, flucytosine, griseofulvin, hachimycin, haloprogin, hydroxystilbamine isethionate, iodochlorohydroxyquinone, isoconazole, itraconazole, ketoconazole, lanoconazole, luflucarban, mepartricin, metroconazole, metronidazole, miconazole, naftifine, natamycin, neomycin, neticonazole, nifuroxime, nystatin, omoconazole, oxiconazole, pentamycin, propionic acid, protiofate, pyrrolnitrin, ravuconazole, saperconazole, selenium sulfide, sertaconazole, sulbentine, sulconazole, terbinafine, terconazole, tioconazole, tolciclate, tolnaftate, triacetin, undecenoic acid, voriconazole, and their pharmaceutically acceptable salts and esters.

17. The formulation of claim 1 wherein the concentration of the carrier is from about 15% to about 50% by weight of the formulation, the concentration of the propellant is from about 15% to about 50% by weight of the formulation, and the concentration of the carrier fluid is from about 10% to about 60% of by weight of the formulation and the concentration.

18. The formulation of claim 13, further comprising one or more pharmaceutically acceptable excipients in a concentration less than about 20% by weight of the formulation.

19. The formulation of claim 1 comprising:
one or more pharmaceutically active agents selected form the group consisting of nystatin, miconazole, clotrimazole, terbinafine, tolnaftate and butenafine, and pharmaceutically acceptable salts and esters thereof,
a carrier for the pharmaceutically active ingredient(s),
a carrier fluid, and
a pharmaceutically-acceptable propellant,
wherein the concentration of the carrier is from about 15% to about 45% by weight of the formulation, the concentration of the carrier fluid is at least about 10% by weight of the formulation, and the concentration of the propellant is from about 20% to about 60% by weight of the formulation, and
wherein the carrier fluid comprises at least about 50% by weight of one or more highly volatile silicones.

20. The formulation of claim 19 comprising a USP-grade nystatin/talc composition having about 100,000 units of nystatin per gram of talc, wherein the concentration of the nystatin/talc composition is from about 15% to about 45% by weight of the formulation.

21. The formulation of claim 19 wherein the propellant is selected from the group consisting of hydrofluorocarbons, alkanes, alkenes, and combinations thereof.

22. The formulation of claim 17 wherein the carrier fluid comprises one or both of hexamethyldisiloxane and octamethyltrisiloxane.

23. The formulation of claim 19, the formulation comprising:
a USP-grade nystatin/talc composition having about 100,000 units of nystatin per gram of talc, wherein the concentration of the nystatin/talc composition is from about 15% to about 45% by weight of the formulation,
a volatile silicone comprising hexamethyldisolaxane, octamethyltrisiloxane, and combinations thereof, wherein the concentration of the volatile silicone is from about 10% to about 35% by weight of the formulation; and
a propellant comprising tetrafluoroethane.

24. The formulation of claim 23 further comprising one or more pharmaceutically acceptable excipients, wherein the concentration of the excipients is less than ten percent by weight of the formulation.

25. The formulation of claim 1 wherein the weight ratio of the pharmaceutical plus the carrier to the carrier fluid plus the propellant is in the range of about 45:55 to about 20:80.

26. The formulation of claim 1 wherein the carrier comprises about 15% to about 45% of the composition, the carrier fluid comprises about 15 to about 40% of the composition, the propellant comprises about 15% to about 40% of the composition, and up to about 10% of the composition comprises excipients, all by weight.

27. The formulation of claim 1 wherein a particulate material is selected from the group consisting of iodine, cadexomer, cadexomer iodine, silver, and pharmaceutically acceptable silver salts.

28. An aerosol container comprising the formulation of claim 1.
